(19) United States
(12) Patent Application Publication
(10) Pub. No.: US 2009/0270690 A1
(43) Pub. Date: Oct. 29, 2009

(54) SYSTEM AND METHOD FOR USING INTERACTIVE VOICE-RECOGNITION TO AUTOMATE A PATIENT-CENTERED BEST PRACTICE APPROACH TO DISEASE EVALUATION AND MANAGEMENT

(75) Inventors: BERNARD A. ROOS, MIAMI BEACH, FL (US); HERMAN S. CHEUNG, MIAMI, FL (US)

Correspondence Address: CHRISTOPHER & WEISBERG, P.A., 200 EAST LAS OLAS BOULEVARD, SUITE 2040, FORT LAUDERDALE, FL, 33301, US

(73) Assignee: UNIVERSITY OF MIAMI, MIAMI, FL (US)

(21) Appl. No.: 12/432,127

(22) Filed: Apr. 29, 2009

Related U.S. Application Data
(60) Provisional application No. 61/048,586, filed on Apr. 29, 2008.

(51) Int. Cl. A61B 5/00; G10L 21/00

(52) U.S. Cl. 600/300; 705/2; 705/3; 704/270.1; 704/E21.001

(57) ABSTRACT

A method and system for monitoring, evaluating, and improving effectiveness and efficiency for treating chronic medical conditions of a large patient population. The inventive method and system utilizes computerized patient interview and data analysis modules for assessing a patient's condition and indicating a need for medical attention. Patient interviews are regularly conducted by telephone using computer generated questions and voice recognition methods to enter responses into a database. A series of medical questions are developed and presented to the patient. Their answers are recorded and analyzed with respect to the database. Based upon the answers and the analysis thereof, a medical action plan is developed, care instructions provided, and an appointment with a doctor scheduled.

### CROSS-REFERENCE TO RELATED APPLICATION

This application is related to and claims priority to U.S. Provisional Patent Application Ser. No. 61/048,586, filed Apr. 29, 2008, entitled SYSTEM AND METHOD FOR USING INTERACTIVE VOICE-RECOGNITION TO AUTOMATE A PATIENT-CENTERED BEST PRACTICE APPROACH TO DISEASE EVALUATION AND MANAGEMENT, the entirety of which is incorporated herein by reference.

### STATEMENT REGARDING FEDERALLY SPONSORED RESEARCH OR DEVELOPMENT

n/a

### FIELD OF THE INVENTION

The present invention relates to a method and system for monitoring, evaluating, and improving effectiveness and efficiency for treating chronic medical conditions of a large patient population.

### BACKGROUND OF THE INVENTION

Individual physicians, medical groups, and hospitals often serve patient populations that can benefit from periodic or regular inquires as to patient health without the patient having to make an office visit. Follow-up care and monitoring is especially important with respect to patients having chronic conditions and/or a history of hospitalizations and emergency room visits to prevent or reduce the likelihood that small, readily-treated and sometimes predictable situations will evolve into dangerous and expensive medical problems.

Presently, other than follow-up visits to a doctor's office, health monitoring to the extent it exists, takes place telephonically whereby a nurse speaks directly with a patient to inquire about the patient's health and perhaps schedules an office visit to be examined by a physician, or perhaps reminds the patient about regular administration of medications. In these live interview situations, competent and knowledgeable nurses are often able to detect potential problems as readily as if a patient were to be physically in the presence of the nurse or a doctor. Then, if a medical problem is identified, the nurse can invite the patient to a doctor's office for a physical assessment and/or laboratory tests. In addition to primary health care providers, many health insurance organizations have programs whereby a staff nurse calls patients in a manner similar to that described above, as a problem detected early will be more effectively and inexpensively treated. Others, such as medical researchers, can also benefit by regular contact with patients.

Although a brief telephone interview can provide distinct clinical, cost, and convenience benefits, such interviews are not employed at anywhere near the frequency that might be reasonable and/or beneficial. The primary obstacle to conducting frequent, large-scale telephone interviews is the aggregate time it takes to conduct such interviews, especially on a large scale. For example, a very large number of interviews (e.g., hundreds or thousands per week), each having a duration of perhaps 5 to 10 minutes per interview, requires hundreds to thousands of nurse/hours per week. Also, additional time is required to review a patient's medical record before the interview, record the substance of the interview, analyze the substance of the interview, determine if medical action is required, and schedule a doctor's office visit or emergency care if appropriate. In situations where a nurse is required to make a large number of calls one after the other, the likelihood that effective review of patient records and analysis thereof in view of the interview responses is diminished. Further, given the shortage of nurses nationwide, it is difficult to obtain sufficient nursing staff, and the cost of implementing such a program, although perhaps cheaper than an office visit, can still be considerable. Yet another complication is the language barrier that exists in an increasingly multi-cultural region where many patients, especially in large urban areas, are either uncomfortable or incapable of clearly communicating in particular language, such as English.

In view of the complexity of dealing with medical conditions on a personalized level with respect to many patients, it would be desirable to integrate particular medical practices with information and communication technologies. An example of such technology is the automated telephone dialer associated with voice query and response software for contacting and querying large numbers of people for the purpose of marketing. Similar systems exist to support customer support, billing, and reservation management. However, as presently known, these systems do not provide the simplicity, security and accuracy required for monitoring chronically ill patients and detecting clinically significant events and/or trends over either a short or a long period of time.

### SUMMARY OF THE INVENTION

The present invention overcomes the above disadvantages of existing methodologies for patient follow-up care by advantageously providing a method and system that makes possible effective, large-scale, low-cost, individualized patient medical follow-up treatment and aftercare using advanced information and communication technology, such as voice query and response systems. While employing sophisticated technology and communication techniques, in addition to highly accurate medical analytics, the system and method of the invention can support patients and health care providers using computers, pagers, and the like as described herein. However, the system can also provide high level support to patients equipped or only comfortable with, an ordinary telephone. The number and content of questions posed to a patient by the system produce a very accurate assessment of a patient's immediate condition or health trend.

The present invention provides a system that is flexible and adaptable, permitting programming of the queries to address any set of standard for evaluation of current status of persons with or at risk for any specified disease(s). The programmability of the system allows modification based upon the chronic illness type and stage. The queries and responses are crafted so as to be simple and to eliminate ambiguity with both the questions and the responses for 100% accuracy; and they can be in any language, such as a patient's preferred spoken language. In response to the queries, artificial intelligence analysis of the system identifies trends and checks and validates past records and makes comparisons to data of other persons with similar chronic conditions. Feedback loops link patient responses to queries to either reassure the patient that his or her condition is progressing normally, not deteriorating, or requiring intervention; and may also generate automated, targeted notifications of as many persons as deemed necessary the care providers and recipients.

In an exemplary embodiment, an automated medical triage tool uses computerized patient interview and data analysis modules for assessing a patient's condition and indicating a need for medical attention. Patient interviews are regularly conducted by telephone using computer generated questions and voice recognition methods to enter responses into a database. The responses obtained over time are analyzed to determine patient condition state and development trend. If the patient's condition appears to be deteriorating, appropriate intervention can be promptly coordinated.

An exemplary method for medical assessment and improved treatment includes creating a database of medical information for a plurality of patients, developing a plurality of medical questions, posing at least one of the plurality of medical questions to at least one of the plurality of patients, recording answers to at least one of the plurality of medical questions from at least one of the plurality of patients, updating the database of medical information to include data from the recorded answers, and analyzing the answers with respect to the database of medical information. Based upon the answers and the analysis thereof, a medical action plan can be developed, care instructions can be provided, and an appointment with a doctor can be scheduled.

In another exemplary embodiment, an interactive medical assessment system is provided. The system includes a database containing medical information related to one or more patients; a processor in electrical communication with the database, the processor for generating one or more medical questions and for recording answers to the medical questions from the one or more patients; and a communication device in electrical communication with the processor, where the communication device is for interfacing over a communication network with one or more selected patients having a compatible communication device and wherein the processor analyzes the answers to the medical questions and updates the database accordingly.

In yet another exemplary embodiment of the invention, a method of medical assessment and treatment is provided. The method includes creating a database of medical information for a plurality of patients, developing a plurality of medical questions related to at least one of a general medical condition and a patient's specific medical history, scheduling an interactive session with a patient, verifying the identity of the patient, using a voice response unit to telephonically pose at least one of the plurality of medical questions to the patient, recording answers to the at least one of the plurality of medical questions from the patient, updating the database of medical information to include data from the recorded answers, and analyzing the updated database to create one or more specific analytical questions.

Further, a method of medical risk management is disclosed that includes posing medical questions to a patient using an automated system over time in a series of discrete sessions; and recording responses from the patient in each of the of discrete sessions. More refined gradation of response and analysis can be achieved through touch-tone input or through expansion of verbal response options from the binary “yes/no” to a scale of “never/occasionally/frequently/always” and the like. These refinements add to the reliability and sensitivity of the trend analysis

Thus, the present invention provides protocols adherent to or surpassing established standards of evaluation of persons with or at high risk for chronic illness-related morbidities and mortality. The information communication technology promotes patient awareness of key factors, educates the patients, and draws the patient into better self-management while facilitating linkage with care providers, thereby reducing an all-too-common sense of isolation. This is accomplished in a manner and schedule adapted to patient needs while freeing patients from the hassle and wasted time of unnecessary visits to possibly distant clinics, the emergency room, or the hospital. The invention reduces the manpower need and cost for evaluation, thereby increasing the efficiency of overall care of a population of chronically ill persons. The net effect is a more effective and efficient care process that is increasingly critical in a world of care provider scarcity.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a schematic illustration of a system for implementing the present invention;

FIGS. 2A and 2B are flow charts that illustrate an exemplary telephonic interview in accordance with the present invention;

FIG. 3 depicts an exemplary assessment script in accordance with the present invention;

FIG. 4 is a flow chart illustrating an exemplary patient exchange that provides reminders to the patient to take their medication and provides educational information

FIG. 5 is a flow chart illustrating an alternate exemplary patient exchange providing reminders to patients to take their medication or to refill their prescriptions; and

FIG. 6 is a flow chart illustrating an exemplary patient exchange that assists patients in maintaining a healthy lifestyle.

### DETAILED DESCRIPTION OF THE INVENTION

The present invention relates to a method of medical assessment and improved treatment and has particular utility in the management of chronic illness such as congestive heart failure (CHF)-HSC-3, asthma, diabetes, and chronic obstructive pulmonary disorder (COPD), allowing “best practice” standards or modified standards to be rigorously implemented. The invention also relates to medical risk management and improved documentation of patient trajectory of chronic illness over time and under various management approaches, as well as improved allocation of medical resources and staffing.

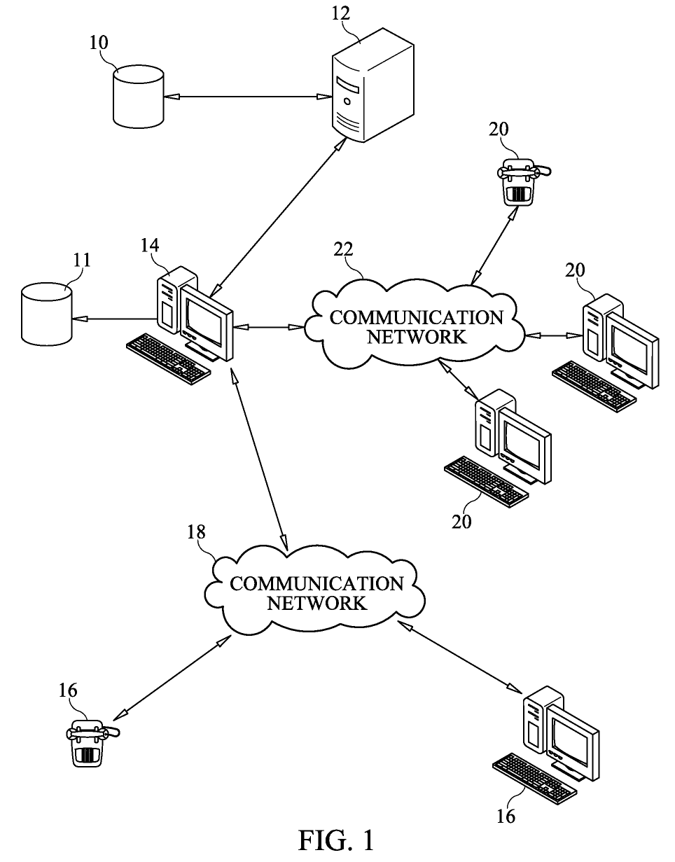

A schematic representation of a system for performing the method of medical assessment and improved treatment is illustrated in FIG. 1, wherein a database 10 has been created that includes data for one or more patients, such as patient identification, related contacts, stored queries and responses, corresponding status reports, care managers, and caregivers. As further described below, the database 10 can include only patient responses to medical questions and a separate database 11 can be provided that includes confidential patient biographical and administrative data in order to comply with HIPPA requirements. The separate database 11 can also be designated to receive status and trend reports. A processing system 12 includes logical systems and analytical algorithms for generating medical, biographical, and administrative questions, as well as processing chronic disease related medical and administrative information. Responses stored in the database 10 can be analyzed by the processing system 12 and information analyzed by the processing system can be stored in the database. Additionally, status or trend reports can be generated.

The processing system 12 is in communication by way of an interactive programmed linkage to a router and information management and distribution system (e.g., phone lines, wireless, cable, Internet, satellite) to a system communications device 14 that is used to interface with one or more selected patients having a compatible patient communication device 16, over a known communication network 18, to enable the at least one medical, biographical, and administrative question to be posed to the patient. Replies to the questions are processed by the processing system 12 and additional patient data is added to the database 10 and database 11 and/or the data within databases 10 and 11 are updated in response to the received patient. The communications device 14 is also capable of communicating with communication devices 20 associated with medical providers, care managers, authorized family members etc., over a known communication network 22. Additional information and/or responses from the communication devices 20 associated with the medical providers, care managers and authorized family members can be established or forwarded to the patient communication device 16 and/or the processing system.

The processing system 12 and the system communications device 14 can include an intelligent automated dialogue system, which may include several components including a natural language processing and understanding module, the capability to process patient speech; a knowledge base and a patient model, the capability to generate appropriate replies; and a natural language generation module that delivers the replies. The patient model and the knowledge base allow customization of the dialogue and implementation of “mixed-initiate” strategies, in which the computer system is able to change the dialogue prompts in response to the quantity and quality of information provided by the patient. The processing system 12 and the communication devices 14 create a chronic disease management network that can pull in as many resources willing to support the care of the patient, subject to the specific standards and methodologies dictated by the professional care team to create a kind of “super care” coordination system. Use of voice responses rather than physiological measurements is a distinctive aspect of the invention in that patient history and regular queries do as well for chronic management as physiological monitoring. Thus, the present invention permits a cheaper and better patient assessment than current technologies. The voice communication approach can be extended to assessing the comfort and reassurance levels of others in the network.

In an exemplary configuration, the processing system 12 is provided with a specific decision support system for one or more medical conditions that correspond to the patient population being monitored. The system accesses the profile for each patient in a database that is progressively updated on the basis of clinical data as well as patient interaction with the system. The system collects information and provides education on symptom monitoring and recognition, adherence to diet, adherence to medications, optimal fluid status and daily physical activity. Although information can be collected, the information may not be shared with others who are in the care network unless the notified care provider does so or formally designates a report from one network or database to another.

Additionally, the system can develop a medical action plan based upon the analysis of the information provided by the patient in conjunction with the information in the database.

Also, as part of the analysis, a notification can be generated that can includes providing medical care instructions. While the notification can be sent to the patient, it can also be sent to one or more of a caregiver, a family member, or a legal guardian. The notification can be a voice message, an electronic text message, an email message, or a printed document, for example.

In the exemplary system, an initial set of medical questions are related to at least one of a general medical condition and a patient's specific medical history, and the system can create additional or follow-up questions based upon analysis of the responses to the questions to create specific analytical questions. These additional answers are then added to the database.

In the exemplary system, the questions are posed and responded to telephonically in voice form which can accurately (100%) be recorded and interpreted for entry into a database and analyzed in terms of past responses and trends. However, electronic text messaging, email, a website, and physical (paper) queries and replies can be incorporated into the system. When the system includes a computer controlled query and response IVR unit and the questions are posed telephonically, the system can verify the identity of the patient before posing medical questions. This can be accomplished with passwords, security questions, voiceprint recognition and/or other known security techniques.

Additionally, the system can use a language (chosen by the patient or the provider) for the voice response unit that corresponds to the language preference of the identified patient or a language from a limited group of languages. The automated query-response system can be implemented in any of several major languages and it can create a world-wide network for evaluation of each of many major diseases. Significantly, the system allows case-control comparisons of various treatment approaches that are known to vary from region to region and among countries and cultures. This allows for very prompt identification of successful and unsuccessful practices by country and region, which then allows for targeted communication and education programs to patients and caregivers.

Additionally, patients can elect the time and frequency of the evaluation call and even active and unscheduled automated evaluation and notification of change that can avoid a “911” call, for which patients and family are often too hesitant to make.

Yet another feature of the invention is the ability of the system to electronically schedule an in-person visit to a selected medical caregiver by interfacing with an electronic appointment calendar or by exchange of messages with the caregiver.

By way of summary, the system automates the most time consuming aspects of disease management (communication and travel time) and affords more efficient targeting and rendering of the human aspects of care by family and professionals.

The invention is also summarized by an exemplary method of medical assessment and treatment having the steps of creating a database of medical information for patients, related to the evaluation, management, and status over time of one or more specific diagnosed medical conditions; developing medical questions related to at least one of general medical condition and a patient's specific medical history; telephonically contacting at least one of the patients; verifying the identity of the at least one patient; using a voice response unit to telephonically pose at least one of the medical questions to the patient; recording answers to the at least one of the medical questions from the patient; updating the database of medical information to include data from the recorded answers; analyzing the updated database to create specific analytical questions; and sending a message to a pre-identified addressee, such as the patient, family, professional care providers, insurers, and other designated by the patient.

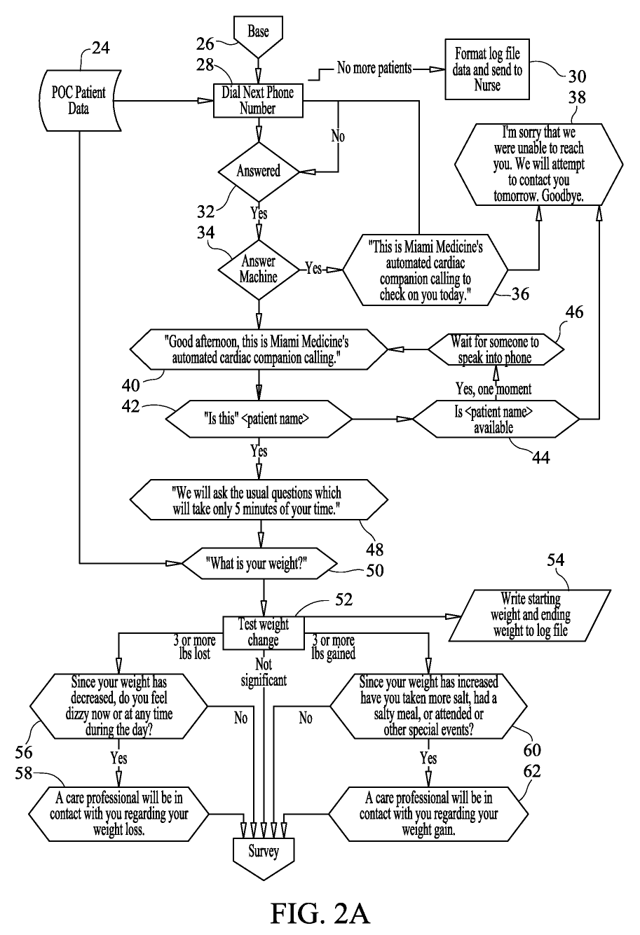

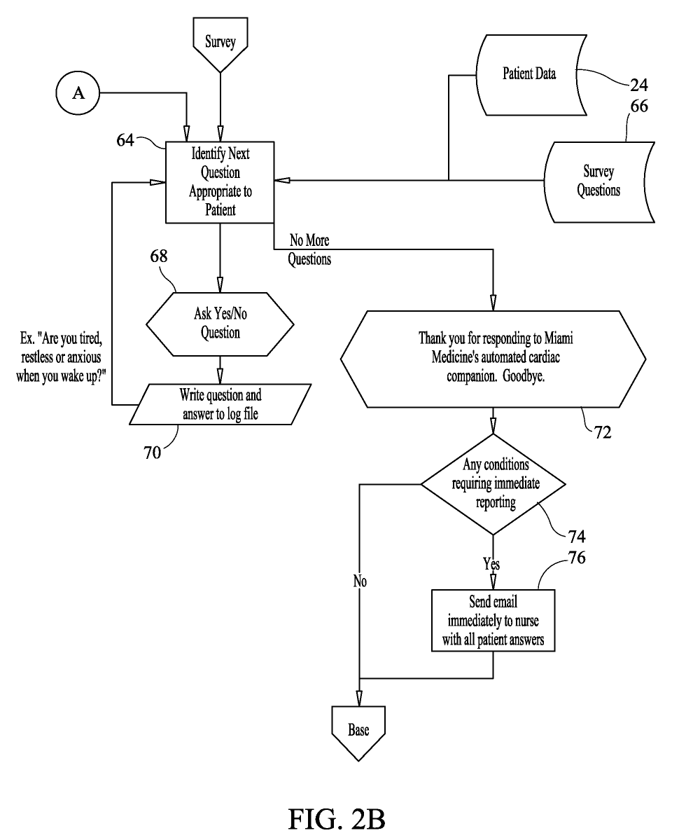

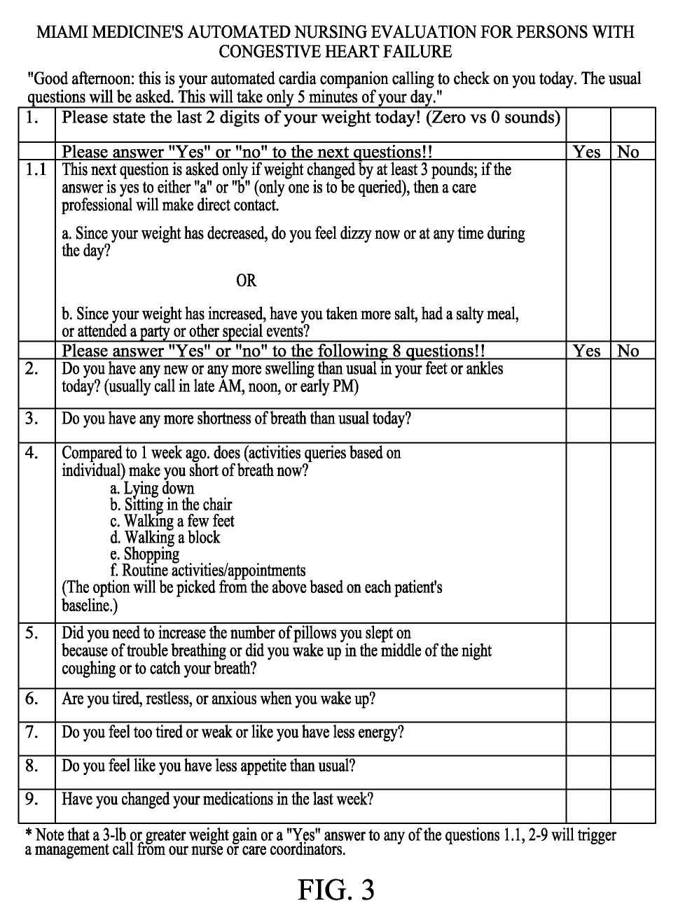

FIGS. 2A and 2B depict a flow chart of steps performed by the system in an exemplary assessment; and FIG. 3 depicts an exemplary assessment script. It will be noted that the assessment script limits responses to very carefully crafted questions to only two choices (typically “yes” or “no”) rather than multiple choices or a point in a range (e.g., 1-9). However, more than two choices can be desirable as voice recognition accuracy improves and by usability determinants of the patient population. The more choices in the response, the fewer encounters it will take to identify trends. In either approach, the questions and responses are crafted to reduce the likelihood of patient overload (remembering more than a few choices can be very difficult) and to remove any possible ambiguity and to eliminate the possibility of misinterpretation of the query or the response. The result is that the accuracy of the assessment is very high unless the patient is mentally incompetent or deliberately evasive. Interestingly, it has been observed that patients are more likely to respond truthfully to sensitive medical questions that are posed with a voice response system than by a live person either telephonically or face-to face. In addition to audio or text questions, graphical choices (e.g., photos or drawings) can be presented for yes/no comparison with the patient's condition (e.g., an ulcer or rash) if the patent has a communication device with a video display, such as a computer screen or smart phone.

It has also been discovered that clinically significant findings can be achieved using a small number of properly crafted questions, for example between 8 and 15 questions. More particularly, it has been discovered that ten properly crafted questions can produce excellent results. Based upon the “yes/no replies, or “never/sometimes/always, or “1/2/3/4/5”, the processing system can provide a simple unambiguous output or message that can be delivered visually or audibly (depending upon the communications equipment used) to the patient, family member or health care provider. In an exemplary system, three states are produced by the analysis: 1) condition red for deteriorating medical condition; 2) condition yellow for a possible problem; and 3) condition green for acceptable medical condition or progress. A condition yellow can invoke a secondary set of decision trees (questions). Simple color codes are readily understood by patients. The simplicity is similarly useful to the health care provider who might monitor for condition yellow and red for timely intervention or human follow-up.

Referring to FIG. 2A, a flowchart of an exemplary interactive communication session between system communication device 14 and a patient communication device 16 can be seen. Utilizing stored patient data 24, a base unit 26 performs the step of dialing the next telephone number on a master list, as per step 28. If the end of the list is reached the log file data is formatted and sent out to a nurse practitioner or other medical assistant, as per step 30. Once the patient's phone is answered at step 32, it is determined if the phone is answered by a person or sent to an answering machine, via step 34. If the call is forwarded to an answering machine, a standard greeting is played, at steps 36 and 38. If the call is received by a person, an automated greeting is played, at step 40. The system than asks the recipient, at step 42, if they are indeed the patient of interest. If they are not, the system is prompted, at step 44, to ask if the actual patient is available. If the patient is available, the system, via steps 46, 40 and 42, once again plays the initial greeting and confirms that the patient is on the other end of the line.

At step 48, an initial prompt is played, which informs the patient that a series of questions are to be asked. The system can ask, for example, what the patient's current weight is, at step 50. By accessing the patient's data 24, which includes initial weights for all patients, the system can compare the patient's current weight to their initial weight. Processor 12 determines whether the patient has gained weight or lost weight, and by how much, via step 52, and the patient's initial and current weights are written to a file, via step 54. Depending upon whether the patient has gained or lost a specific amount of weight, i.e., three pounds or more, a series of questions can then be posed. For example, if the patient has lost weight, the system poses a series of questions to the patient to try and determine the reason for the weight loss, at step 56, and informs the patient that a health care professional will contact them regarding their weight loss, at step 58. Similarly, if the system determines that the patient has gained weight, questions directed toward the possible reasons for the patient's weight gain are presented, at step 60, and informing the patient that a health care professional will contact them regarding their weight gain, at step 62.

If the weight loss or gain is not significant, i.e., less than three pounds, or after the questions about their weight loss or gain are presented to the patient, the system continues its survey by identifying the next appropriate question to ask to the patient, at step 64, as shown in FIG. 2B. Referring to FIG. 2B, these questions can be derived from a series of survey questions 66, utilizing the patient data 24. The series of survey questions 66, typically “yes/no” questions 68, are each written to a log file along with the patient's corresponding answer, via step 70. The survey questions can be tailored to each patient depending upon information in the patient database 24.

Once the survey questions are completed, the generated computer voice thanks the patient, via step 72, and determines if there are any actions to take depending upon the patient's answers, at step 74. For example, if the patient indicated that they were short of breath, and anxious throughout the day, the system might determine that immediate medical care is needed. This determination utilizes previous answers from that patient and stored patient data, including the patient's medical condition. If immediate action is required, the patient's answers are forwarded to a nurse or other health practitioner, at step 76, where a determination on the type of medical care needed for the patient can be made. The scenario presented in FIGS. 2A and 2B is exemplary only and it is within the scope of the present invention to initiate a patient interactive session regarding any patient issues.

FIG. 3 depicts an exemplary assessment script in accordance with the present invention. As shown in steps 40 through 72 in FIGS. 2A and 2B, a series of questions are posed to the patient. Depending upon the patient's response, a different series of questions are presented. The questions are typically of the Yes/No variety but the questions can also depend upon each patient's specific database and can include a choice of answers. For example, step 4 in FIG. 3 poses the question regarding whether or not the patient experiences shortness of breath when engaging in one of several activities (e.g., lying down, sitting in a chair, shopping, etc.). These questions can be selected based upon the patient's medical condition, medical history, and the patient's answers to previous questions. Patients might also be asked to rate their overall medical condition after performing one of several activities. Thus, the assessment script can be tailored to each patient depending upon information stored under that patient's name in databases 10 and 11.

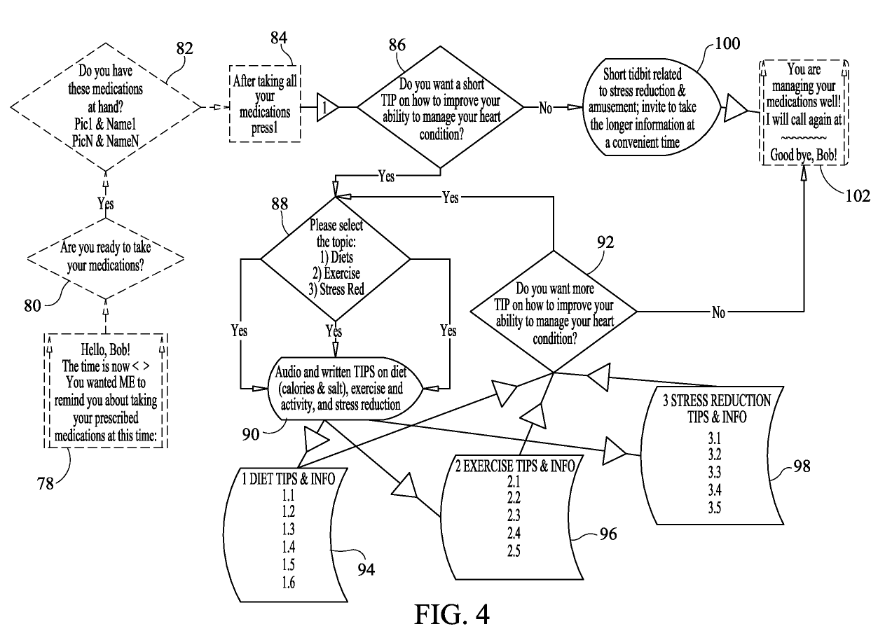

In FIG. 4, a flow chart illustrating an exemplary patient exchange that reminds the patient to take their medication and provides educational information, is shown. After establishing a communication channel with a particular subscribing patient, the system, via a computer-generated voice if the communication is to be an audio communication, identifies the patient by name, and states that the system is reminding the patient that it is time to take their medication, at step 78. The system then asks the patient if they are ready to take their medication, via step 80, and if the answer is “yes”, asks if the patient has their medications at hand, via step 82. The system can account for the scenario where the patient must take more than one type or dose of medication, and will prompt the user to take each of the required medications.

The patient is prompted to indicate when they have taken all of their medication, at step 84, for example, by depressing a number on their phone's keypad. The system then asks if the patient would like to receive a short tutorial on how to manage the patient's current medical condition. Based on the patient's records stored in databases 10 and 11, the system tailors its question to the patient's particular ailment or ailments. For example, the patient may be suffering from a heart condition and, via steps 86 through 100, the patient is presented with a series of informative tips on how to manage their heart condition. Specific tips on dieting, exercise, and stress reduction are presented to the patient depending on what the patient wants to hear at the time. A final system prompt can provide the patient with reassurance that they are indeed attending to their medical condition and should expect a future call, at step 102.

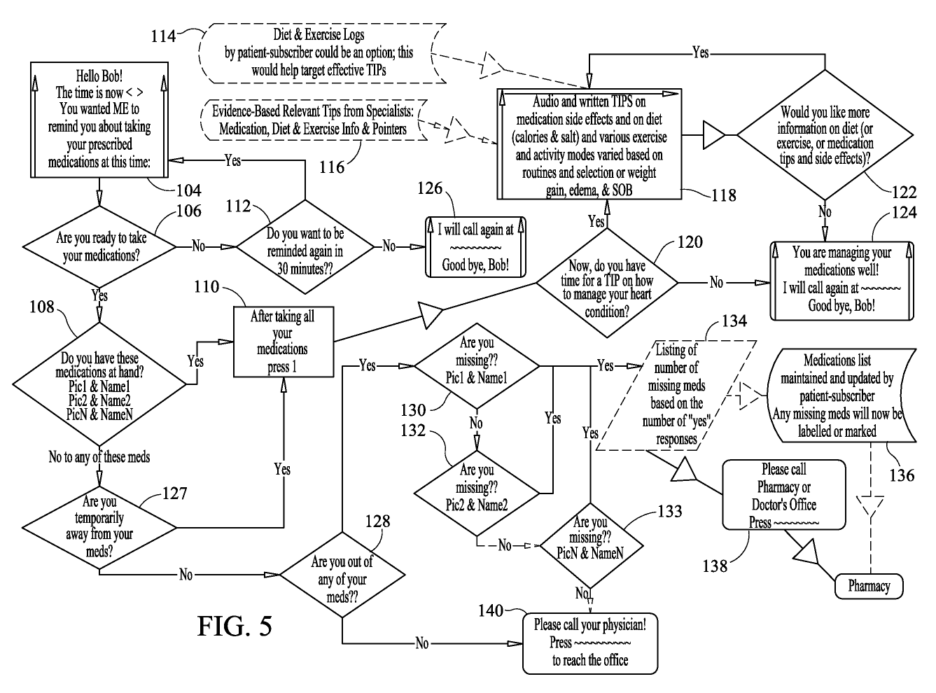

In FIG. 5, a flow chart illustrating an alternate exemplary patient exchange providing reminders to patients to take their medication or to refill their prescriptions, is shown. Similar to the patient exchange illustrated in FIG. 4, steps 104 through 126 walk the patient through a series of interactions where the patient is reminded to take each of their required medications and, if they choose, are presented with a series of tips regarding their medical condition. In the instance where the patient does not have their present medication at hand, the system inquires if the patient is temporarily away from their medication, at step 127. Assuming the patient then retrieves their medication, the system resumes its inquiries with step 110. However, if the patient indicates they are not temporarily away from the medication, the system then inquires if the patient is out of their medication, at step 128. After a series of interactive questions and responses via steps 130 through 136, the system determines if the patient needs to visit a pharmacy to replenish one or more medications, at step 138. The system can list and store the number and names of the missing medication based on the patient's responses, via step 134. Alternatively, the system may determine that the patient needs to contact their physician to discuss obtaining a new prescription, via step 140. Thus, the patient is instructed on the best course of action and is directed to contact their physician or pharmacy depending on the factors of the present situation.

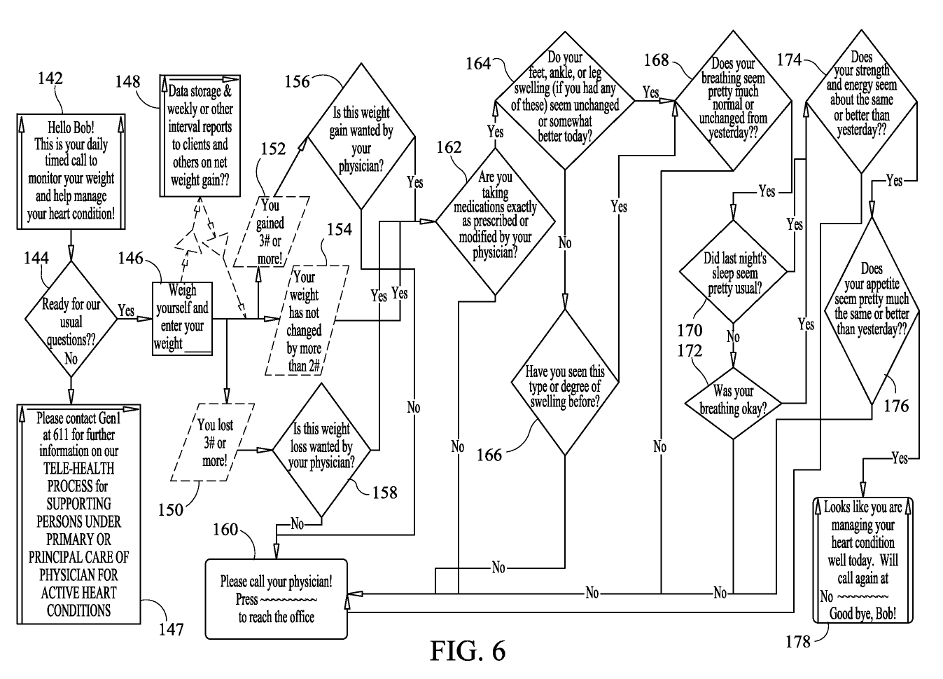

In FIG. 6, a flowchart illustrating an alternate exemplary embodiment of the invention is shown whereby the system, via an interactive exchange with a patient, assists the patient in maintaining a healthy lifestyle. FIG. 6 illustrates another embodiment of the invention yet utilizes the similar voice prompting and interactive session as discussed in the earlier embodiments. In this instance, via step, 142, the system contacts the patient on their communication device and states the nature of the call, in this case, a scheduled call to monitor the patient's weight due to their present medical condition, for example, a heart condition. After determining if the patient is ready, via step 144, the system asks the patient to weigh themselves and then enter their weight via the digital keypad on their communication device, via step 146. Of course, this could also be accomplished by the entering of the weight on the patient's computer or PDA device. The system accounts for instances when the patient may not be ready for the session due to some unforeseen circumstance and directs the patient, via step 147, to contact another information source, i.e., a website, or another phone number, at a more convenient time.

When the patient enters their current weight, the system can perform a series of data storage routines and sub-routines based upon a comparison of the patient's current weight to their previous or target weight. For instance, at step 148, the patient's current weight is stored under their data profile and a report can be generated and presented to the patient so the patient can see their progress. If the patient's current weight exceeds a certain threshold amount, a report can be presented to the patient that discusses the danger of weight-gain for patients with heart conditions. These reports can be presented to the patient in a variety of formats, including a voice recording to the patient's communication device, or written reports provided via email. The invention is not limited to the type, frequency or mode of delivery of such reports. Advantageously, the reports can be tailored to not only weight-related issues, as is illustrated in this embodiment, but to diet, exercise, etc. The system advantageously provides different feedback depending upon the data input by the patient on their communication device during the interactive session and the result of the comparison of the data to that patient's stored medical information.

Referring again to the embodiment depicted FIG. 6, steps 150, 152 and 154 are statements that are communicated to the patient after they have entered their current weight and that amount is compared to their previous entry at an earlier session. If there has been a weight gain or loss (or if there has been a weight gain or loss of more than a threshold amount), the system, via steps 156, 158, asks the patient if the weight gain or loss was desired by the patient's physician. If the weight gain or loss was not expected, the system prompts the patient to contact their physician, at step 160. If the weight gain or loss was expected, the system then asks the patient if they are taking their medication exactly as prescribed, at step 162.

Assuming the patient is taking their medications exactly as prescribed (if they are not, they are prompted to contact their physician via step 160), a series of questions are presented that focus on any unusual changes in the patient's physical well-being, e.g. feet, ankle, or leg swelling, breathing difficulty, overall strength and energy level, sleep habits, and appetite, via steps 164 through 176. If the patient identifies any problems in their well-being, the system prompts them to contact their physician, at step 160. Otherwise, at step 178, the system reassures the patient that they are managing their condition in a satisfactory way.

The questions presented by the system of the present invention are tailored to the patient's current or past medical condition and let the patient know that they will be contacted at the next scheduled time. The questions may vary from session to session depending upon the patient's answers in prior sessions. Thus, the invention advantageously provides a scheduled, informative and dynamic interactive health monitoring session tailored to the patient over a normal communication channel, i.e., the patient's cellular phone, land-line, personal computer, or PDA.

A feature of the present system is its capability to perform trend analysis of the patient's replies. Depending upon the medical condition and the treatment regimen, the answers to certain questions are expected to change over time and others are not. If the expected answers do not match the profile for an expected outcome, a condition yellow or red can be generated. Further, the perfect accuracy of record keeping and trend analysis from perhaps 10 to 20 or more assessments over hours, days or months permits an assessment to be performed within a short timeframe that is far more likely to identify items of medical significance earlier than would likely be achieved by any other care approach, including a similar interview conducted by a “live” person. For example, the number of “bad days” that a patient reports in an assessment, while subjective, is a subtle and important health indicator. Patients would not be likely to record such information and bring it to an in-person interview at the doctor's office. Trying to recall such impressions from memory with any accuracy over even a short period is virtually impossible. In short, the system can do what even a top medical staff cannot.

Implementation of the above combination of IVR, database documentation and trend analysis, plus reporting to target persons who need attention, by providers and family, thus provides critical, accurate and timely feedback from patients so that unnecessary hospital, emergency room and doctor visits can be reduced. Significantly, rather than, for example, three somewhat random post hospitalization visits to assess recovery/healing, monitoring of a patient can be adjusted to the expected rate of recovery for the particular disease and patient, so that office or home visits, or nurse or other professional interactions are only scheduled if actually appropriate. In addition to the resultant cost saving and increase in caregiver efficiency, patient and family satisfaction and quality of life are improved and documentation is clearer for both treatment and risk management.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of medical assessment and treatment, comprising:
creating a database of medical information for a plurality of patients;
developing a plurality of medical questions;
posing at least one of the plurality of medical questions to at least one of the plurality of patients;
recording answers to at least one of the plurality of medical questions from at least one of the plurality of patients;
updating the database of medical information to include data from the recorded answers; and
analyzing the answers with respect to the database of medical information.

2. The method of claim 1, further comprising developing an action plan based at least in part upon the analyzing of the answers.

3. The method of claim 1, further comprising selecting a health state selected from at least three distinguishable health states defined by the analyzing of the answers.

4. The method of claim 1, further comprising generating a notification based upon the analyzing of the answers.

5. The method of claim 4, wherein generating a notification includes providing at least one of a plurality of patients with medical care instructions.

6. The method of claim 4, wherein generating a notification includes sending a message to a pre-identified addressee.

7. The method of claim 6, wherein the pre-identified addressee includes at least one of a caregiver, a family member, a legal guardian, patient representative, and person designated or approved by the patient or the patient's legal guardian or representative.

8. The method of claim 4, wherein generating a notification includes creating at least one of a voice message, an electronic text message, an email message, a printed document, a visual signal, a color-coded signal, and a change of state of a remote electronic device.

9. The method of claim 1, wherein the medical questions include aggregating questions related to at least one of a general medical condition and a patient's specific medical history.

10. The method of claim 9, further comprising analyzing the responses to questions related to the general medical condition and the patient's specific medical history to create one or more specific analytical questions.

11. The method of claim 1, further comprising adding the recorded answers to the database of medical information.

12. The method of claim 1, further comprising posing questions via at least one of telephonically, using electronic text messaging, via email, via a website, or on paper.

13. The method of claim 12, wherein a computer controlled interactive voice response unit poses questions telephonically.

14. The method of claim 1 further comprising verifying the identity of the patient prior to posing at least one of the plurality of medical questions.

15. The method of claim 1, further comprising electronically scheduling a direct interactive communication with a selected medical care giver.

16. An interactive medical assessment system comprising:
a database containing medical information related to a plurality of patients;
a processor in electrical communication with the database, the processor for generating a at least one of the plurality of medical questions and for recording at least one answer to the medical questions from the one or more patients; and
a communication device in electrical communication with the processor, the communication device for interfacing over a communication network with one or more selected patients having a compatible communication device;
wherein the processor analyzes the answers to the medical questions and updates the database accordingly.

17. The system of claim 16, further comprising an automated dialogue system including a voice generation and voice recognition module.

18. A method of medical assessment and treatment, comprising:
creating a database of medical information for a plurality of patients;
developing a plurality of medical questions related to at least one of a general medical condition and a patient's specific medical history;
scheduling an interactive session with a patient;
verifying the identity of the patient;
using a voice response unit to telephonically pose at least one of the plurality of medical questions to the patient;
recording answers to the at least one of the plurality of medical questions from the patient;
updating the database of medical information to include data from the recorded answers; and
analyzing the updated database to create one or more specific analytical questions.

19. The method of claim 18, further comprising sending a message to a pre-identified addressee.

20. The method of claim 18, wherein the medical questions posed can only be answered in a binary form.

21. The method of claim 18, wherein the answers to the at least one of the plurality of medical questions are subjected to trend analysis to determine a health state, the health state selected from at least three distinguishable health states.

22. The method of claim 21, wherein one health state represents a bad or deteriorating health condition.

23. The method of claim 21, wherein one health state represents a health condition requiring further investigation.

24. The method of claim 21, wherein one health state represents an acceptable or improving health condition.
